(19) United States
(12) Patent Application Publication
(10) Pub. No.: US 2006/0178349 A1
(43) Pub. Date: Aug. 10, 2006

(54) Compositions and therapeutic methods utilizing a combination of a 5-HT1F inhibitor and an NSAID

(75) Inventor: John R. Plachetka, Chapel Hill, NC (US)

Correspondence Address: FITCH, EVEN, TABIN & FLANNERY, P. O. BOX 65973, WASHINGTON, DC, 20035, US

(73) Assignee: POZEN Inc., Chapel Hill, NC (US)

(21) Appl. No.: 11/332,795

(22) Filed: Jan. 17, 2006

Related U.S. Application Data
(60) Provisional application No. 60/645,599, filed on Jan. 24, 2005.

(51) Int. Cl. A61K 31/4545; A61K 31/60; A61K 31/415; A61K 31/405; A61K 31/365; A61K 31/192

(52) U.S. Cl. 514/165; 514/318; 514/420; 514/406; 514/471; 514/569; 514/570

(57) ABSTRACT

The present invention is directed to compositions containing a 5-HT_{1F}-specific agonist that acts by blocking protein extravasation together with an NSAID. These compositions may be used to treat migraine and headache pain. The invention also includes methods in which these drugs are separately administered to a patient.

### CROSS REFERENCE TO RELATED APPLICATIONS

The present application claims priority to, and the benefit of, U.S. provisional application 60/645,599, filed on Jan. 24, 2005. The contents of this prior application are hereby incorporated by reference in its entirety.

### FIELD OF THE INVENTION

The present invention is directed to pharmaceutical compositions that contain a 5-HT_{1F} agonist (an anti-inflammatory agent that acts by preventing protein extravasation) together with a nonsteroidal anti-inflammatory drug (NSAID). The pharmaceutical compositions may be administered to relieve headache pain and will be particularly useful in treating migraine.

### BACKGROUND OF THE INVENTION

5-HT_{1B/1D}-specific triptans (e.g., sumatriptan) are often used in treating migraine because of their relatively rapid onset of action and long duration of effectiveness. These triptans bind selectively to 5-HT_{1B} and 5-HT_{1D} serotonin receptor subtypes and, to a lesser extent, to 5-HT_{1F} receptors (see generally, Goadsby, et al., Neuroscience 122:491-498 (2003)). The mechanism by which the triptans act is somewhat controversial, but their initial effects may be due, at least in part, to an ability to constrict cranial blood vessels (Saxena, et al., Trends Pharmacol. Sci. 10:200-204 (1989); Humphrey, et al., Br. J. Pharmacol. 94:1123-1132 (1994)). Unfortunately, these triptans may also cause coronary or pulmonary arteries to constrict and this can lead to side effects such as elevated blood pressure, myocardial ischemia, dizziness, tingling sensations and chest pain. As a result, the 5-HT_{1B/1D}-specific triptans are contraindicated in patients with heart disease (Plosker, et al., Drugs 47:622-651 (1994); Ottervanger, et al., CNS Drugs 3:90-98 (1995); Luman, et al., Lancet 341:1091-1092 (1993)).

The 5-HT_{1B/1D}-specific triptans may also relieve pain by reducing cranial inflammation. This view is supported by studies showing that the triptans block neuropeptide release and dural plasma protein extravasation (Buzzi, et al., Cephalalgia 15:277-280 (1995); Buzzi, et al., Pathol. Biol. 40:313-317 (1990)). The anti-inflammatory action appears to be due to the binding of the triptans to 5-HT_{1F} receptors (Johnson, et al., NeuroReport 8:2237-2240 (1997)), whereas vasoconstriction appears to be caused by binding to 5-HT_{1B} receptors (Verheggen, et al., Br. J. Pharmacol. 124:1345-1354 (1998); DeVries, et al., Br. J. Pharmacol. 127:405-412 (1999)). These findings have led scientists to conclude that compounds binding exclusively to 5-HT_{1F} receptors, and other compounds that are active in models of cranial inflammation but which do not induce vasoconstriction (e.g., NK-1 antagonists), might relieve migraine pain without the side effects described above. However, clinical studies using drugs specific for neurogenic inflammation have often produced poor results in terms of efficacy (Goldstein, et al., Lancet 358:1230-1234 (2001); Ramadan, Curr. Med. Res. Opin. 17(suppl. 1):S75-S80 (2001); Roon, et al., Ann. Neurol. 47:238-241 (2000); Goldstein, et al., Clin. Pharmacol. Ther. 67:419-426 (2000); Goldstein, et al., Cephalalgia 17:785-798 (1997); Goldstein, et al., Cephalalgia 21:102-106 (2001); and May, et al., Expert Opin. Investig. Drugs 10:673-678 (2001)).

A different group of drugs that has been used to treat pain, including migraine pain, is the nonsteroidal anti-inflammatory drugs (NSAIDs). Unlike 5-HT_{1F} agonists, it is believed that the primary action of the NSAIDs is to block the activity of cyclooxygenase (COX) enzymes, thereby inhibiting the production of pro-inflammatory prostaglandins and providing effective treatment of existing inflammation. In general terms, NSAIDs are advantageous for many conditions because they have been proven effective in alleviating existing symptoms.

### SUMMARY OF THE INVENTION

The present invention is based upon the concept that NSAIDs and agents that reduce inflammation by inhibiting protein extravasation, in particular 5HT_{1F} agonists, have a complementary effect in relieving pain, particularly migraine pain. The NSAIDs will act to block or neutralize the effects of pro-inflammatory agents that have already leaked from blood vessels, whereas agents reducing extravasation will block the further release of pro-inflammatory substances. Thus, the NSAIDs can provide a rapid relief of pain and both the NSAIDs and extravasation inhibitors will act together to provide longer term relief by stopping the extension of inflammation caused by the release of additional inflammatory substances.

In its first aspect, the invention is directed to a pharmaceutical composition in unit dose form which contains an anti-inflammatory compound that is a 5-HT_{1F} agonist and that acts by blocking protein extravasation (e.g., as determined using the assay of Markowitz, et al. (J. Neurosci. 7:4129-4136 (1987)) together with an NSAID. Each of these drugs should be present in an amount effective to relieve pain upon the administration of one or more of the unit doses to a patient, and a synergistic effect should generally be evident at doses of the extravasation blocker that appear to provide little or no clinical benefit when used alone.

The most preferred 5-HT_{1F} agonists are described in international application PCT/US0308455 as having the structure:

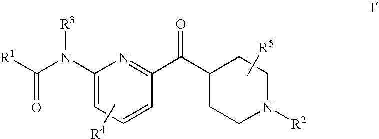

wherein: R¹ is a C₁-C₆ alkyl, C₃-C₇ cycloalkyl, substituted C₃-C₇ cycloalkyl, C₃-C₇ cycloalkyl-C₁-C₃ alkyl, substituted C₃-C₇ cycloalkyl-C₁-C₃ alkyl, phenyl, substituted phenyl, heterocycle or substituted heterocycle,

R² is H, C₁-C₃ alkyl, C₃-C₆ cycloalkyl-C₁-C₃ alkyl, or a group of formula II′;

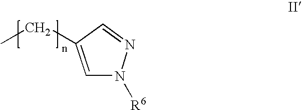

R³ is H, or a C₁-C₃ alkyl;

R⁴ is H, halo, or C₁-C₃ alkyl;

R⁵ is H, or a C₁-C₃ alkyl;

R⁶ is H or C₁-C₆ alkyl;

and n is an integer from 1 to 6 inclusively.

The term “halo” as used above in reference to structures I′ and II′ refers to fluoro, chloro, bromo or iodo. “Heterocycle” refers to a saturated or unsaturated 5 or 6 membered ring containing from 1 to 3 heteroatoms selected from nitrogen, oxygen and sulfur with the ring optionally being benzofused. Examples of heterocycles include pyridinyl, indolyl, furanyl, benzofuranyl, thiophenyl, benzoioxolyl and thiazolidinyl, all of which may optionally be substituted. The term “substituted” when used in connection with alkyl, cycloalkyl, cycloalkyl, alkoxy or alkylthio groups means that these groups may be substituted one or more times independently with a substituent selected from the group consisting of halo, hydroxy and C₁-C₃ alkoxy. For example, substituted groups would include: trifluoromethyl; pentafluoroethyl; 3-hydroxypropyloxy; 5-fluoro-2-bromopentyl; 3-hydroxypropyloxy; chlorocylohexyl etc. The terms “substituted phenyl” and “substituted heterocycle” mean that cyclic moieties are substituted with one or more substituents independently selected from the group consisting of: halo; C₁-C₄ alkyl; C₁-C₄ alkoxy; and C₁-C₄ alkylthio; where each alkyl, alkoxy and alkylthio substituent can be further substituted with C₁-C₂ alkoxy, with one to five halo groups selected from fluoro and chloro, or with one substituent selected from the group consisting of phenyloxy, benzyloxy, phenylthio, benzylthio, and pyrimidinyloxy. The phenyloxy, benzyloxy, phenylthio, benzylthio, and pyrimidinyloxy substituents can be further substituted with one or two substituents selected from the group consisting of halo, C₁-C₂ alkyl, and C₁-C₂ alkoxy or substituted with one substituent selected from the group consisting of C₁-C₄ acyl, and C₁-C₄ alkoxycarbonyl. Further substitution of compounds may include one substituent selected from the group consisting of halo, C₁-C₄ alkyl, C₁-C₄ alkoxy and C₁-C₄ alkylthio.

It will be understood that any pharmaceutically acceptable salt of a 5-HT_{1F} agonist is suitable for use in the pharmaceutical compositions of the invention. Similarly, prodrugs and esters of the agonists that release the agonist after ingestion should be considered substantially the same as the agonists themselves. Overall the single most preferred 5-HT_{1F} agonist has the structure of formula I′ where: R¹ is a 2,4,6-trifluorophenyl group, R² is a methyl and R³, R⁴, and R⁵ are all hydrogen. Thus, the most preferred compound is 2,4,6-trifluoro-N-[6-(1-methyl-piperidin-4-ylcarbonyl)-pyridin-2-yl]-benz amide along with its pharmaceutically acceptable salts.

NSAIDs that can be used include: aspirin; ibuprofen; acetaminophen; naproxen; diclofenac; ketorolac; etodolac; flurbiprofen; oxaprozin; indomethacin; mefenamic acid; nabumetone; piroxicam; celecoxib; rofecoxib; valdecoxib; and lomoxicam. These should, in general, be present in an amount of between 1 and 600 mg, whereas the 5-HT_{1F} agonist should generally be present in an amount of between 0.5 and 600 mg, and preferably at between 10 and 400 mg. These drugs may also be present in an amount sufficient to provide for therapeutic synergy or migraine-related therapeutic synergy, as these terms are defined herein. The most preferred NSAIDs for use in pharmaceutical compositions are: aspirin; ibuprofen; acetaminophen; naproxen; diclofenac; ketorolac; lornoxicam; and etodolac. Unless otherwise indicated, it is intended that any pharmaceutically acceptable form or salt of a drug referred to herein may be used in compositions and methods, and that the weights provided refer to the free form of the drug.

In another aspect, the present invention is directed to methods for treating a patient for migraine by administering a therapeutically effective amount of any of the pharmaceutical compositions described above. The invention also includes treatment methods in which a patient is administered a therapeutically effective amount of an NSAID and separately administered a therapeutically effective amount of a 5-HT_{1F} agonist. The NSAID and 5-HT_{1F} agonist should be administered in a cotimely manner, i.e., they should be administered in close enough temporal proximity that their therapeutic effects overlap. Preferably, the administration of the 5-HT_{1F}-specific agonist and NSAID occurs within two hours of one another. The most preferred compounds and preferred dosages are the same as those discussed above in connection with pharmaceutical compositions.

This procedure may be used to treat headaches falling into a wide variety of classes including: migraine headache; tension-type headache; cluster headache and chronic paroxysmal hemicrania; miscellaneous headache unassociated with a structural lesion; headache associated with a non-vascular intracranial disorder; headache associated with the administration of a substance or its withdrawal; headache associated with noncephalic infection; headache associated with a metabolic disorder; headache associated with a disorder of the cranium, neck, eyes, ears, nose, sinuses, teeth, mouth or other facial or cranial structure; cranial neuralgias; and nerve trunk pain and deafferentiation pain. (For a description of classes, see Olesen, et al., The Headaches, pp. 9-14, Raven Press; see also, “Classification and diagnostic criteria for headache disorders, cranial neuralgias and facial pain,” Headache Classification Committee of the International Headache Society, Cephalalgia 8(supp. 7):1-96 (1988)).

In cases where patients are treated for migraine, the administration of NSAID may be preceded by a step in which a gastric prokinetic agent, preferably metoclopramide, is given to the patient at a dose of between 5 and 40 mg. In general, it is preferred that the metoclopramide be given between 10 and 30 minutes prior to NSAID, but the drugs may also be given concurrently if desired. Alternatively, metoclopramide may be included as part of unit dosage forms which may be, optionally, coordinated to release the metoclopramide first (see US U.S. Pat. No. 6,479,551).

### DETAILED DESCRIPTION OF THE INVENTION

I. Definitions

A. “Unit dose” or “unit dosage form” refers to a single drug administration entity. By way of example, a tablet or capsule containing an extravasation inhibitor and an NSAID would be a unit dosage form.

B. “5-HT_{1F}-specific agonist” or “5-HT_{1F} agonist” refers to a ligand that binds to the 5-HT_{1F} receptor with greater affinity than to the other serotonin receptor subtypes and which, upon binding, mimics the effects of the binding of the endogenous ligand. For the purposes of the present invention, 5-HT_{1F}-specific agonists should have at least a 10-fold greater affinity for the 1F receptor subtype than for the 1B subtype. Preferably, there should be at least a 30-fold greater affinity and, more preferably, at least a 300-fold greater affinity. The 5-HT_{1F} receptors are found primarily within the CNS and, as discussed further below, many 5-HT_{1F}-specific agonist compounds have been described in the art.

C. “Migraine” refers to a well known medical condition characterized by recurrent severe headache that is often accompanied by other symptoms such as nausea, vomiting and heightened sensitivity to light or sound. Patients sometimes have an “aura” that immediately precedes an attack during which they may experience alterations in vision, flashes of light or numbness. Migraine symptoms often subside upon treatment and then recur as a “relapse headache” within 48 hours.

D. “NSAIDs” refers to a group of nonsteroidal anti-inflammatory drugs that are well recognized in the art as analgesics and that act by inhibiting the activity of cyclooxygenase. In this way, the NSAIDs prevent the generation of pro-inflammatory prostaglandins. Acetaminophen also inhibits prostaglandin synthesis, but exhibits weak activity against the cyclooxygenase enzymes. Although the art does not generally recognize acetaminophen as an NSAID, unless otherwise indicated, it will be considered as an NSAID for the purposes of the present invention.

E. “Therapeutically effective” refers to a dosage of a drug or combination of drugs that provides the specific pharmacological response for which the drug or drugs have been administered in a significant number of subjects in need of such treatment. Thus, a therapeutically effective amount of an anti-inflammatory drug is a dosage sufficient to reduce the swelling or pain associated with inflammation. Similarly, a therapeutically effective dose of a drug administered to treat migraine would be an amount sufficient to reduce the pain or other symptoms that are associated with migraine.

F. “Onset of action” refers to the interval that begins when a drug is first ingested by a patient and that ends when a therapeutic effect is first observed.

G. “Therapeutic synergy” for a combination of a 5-HT_{1F} agonist and an NSAID means that, as measured in a population of patients, the combination exhibits one or more of the following: a) a longer duration of pain relief than that achievable using either drug alone, i.e., as the sole active agent; b) a greater reduction in pain severity than is achievable by the administration of either drug alone; and c) a reduction in one or more undesirable side effects associated with the administration of either drug alone. Among the side effects that may be reduced are: dizziness, gastrointestinal lesions, blood clots, stroke, heart attacks, ulcers, heart palpitations and discomfort caused by the constriction of blood vessels. When referring to migraine, the term “migraine-related therapeutic synergy” is used. This has the same definition as “therapeutic synergy,” except that an additional characteristic that may be indicative of synergy is: d) a reduction in the frequency of a relapse headache that is greater than that achievable using either drug by alone.

H. “Anti-inflammatory compounds that act by blocking protein extravasation” are compounds showing activity in animal models of inflammation that measure the leakage of protein from blood vessels. In this regard, it should be noted that vasodilation and increased vascular permeability are two of the main characteristics that are associated with the inflammatory response. One inflammation assay that is accepted in the art and that measures protein extravasation has been described by Markowitz, et al. (J. Neurosci. 7:4129-4136 (1987)). This assay has been used both for 5-HT receptor agonists and for NK-1 antagonists (see May, et al., Curr. Opin. Neurol. 14:341-346 (2001)). As used herein, the term is intended to exclude NSAIDs.

I. “Cotimely” with respect to drug administration refers to the administration of a second drug for the treatment of a condition while a first drug is still present in a therapeutically effective amount.

J. “Coordinated” or “coordinated drug release” as used herein refers to the orderly, sequential release of drug agents from a dosage form. When referring to a composition containing a gastric prokinetic agent, an NSAID and a 5-HT_{1F}-specific agonist, a coordinated dosage form would release the gastric prokinetic agent first and the other drugs afterwards. More specifically, at least 80% of the total gastric prokinetic agent present in the dosage form should be released into the gastrointestinal tract at a time when less than 20% of NSAID or other anti-inflammatory compound has been released. The term can also be applied to a drug composition in which an NSAID and a 5-HT_{1F}-specific agonist are present without metoclopramide. In this case, at least 80% of the NSAID should be released at a time when less than 20% of the 5-HT_{1F}-specific agonist is released, or vice versa.

K. “Multilayer tablet” refers to a tablet dosage form in which components are found in two or more distinct regions. For example, a multilayer tablet may contain an outer layer in which NSAID is essentially the only active agent and an inner layer in which essentially the only active agent is an 5-HT_{1F} agonist.

II. Therapeutic Agents

A. NSAIDs

NSAIDs compatible with the present invention are well known in the art and are either commercially available or can be synthesized using standard techniques of medicinal chemistry. Although the dosage of NSAID may be adjusted by a clinician on a case-by-case basis, general guidelines have been established in the art for many of these compounds.

Examples of NSAIDs (with typical daily doses in parentheses) are as follows: propionic acids (fenoprofen (1500 mg); flurbiprofen (200 mg); suprofen; benoxaprofen; ibuprofen (1600 mg); ketoprofen (200 mg); naproxen (750 mg); oxaprozin (1200 mg)); acetic acids (diclofenac (100 mg); aceclofenac (200 mg); etodolac (1200 mg); indomethacin (75-150 mg); ketorolac (10-30 mg)); ketones (nabumetone (1500 mg); sulindac (300 mg); tolmetin (800 mg)); fenamates (meclofenamate (400 mg); tolfenamic acid (400 mg); mefanamic acid); oxicams (droxicam; piroxicam (20 mg); lornoxicam (30 mg); meloxicam (15 mg); tenoxicam) salicylates (aspirin; diflunisal); pyrazolinates (oxyphenbutazone; azapropazone; phenylbutazone); COX-2 inhibitors (rofecoxib (50 mg); valdecoxib (20-40 mg); etorocoxib (60-120 mg); celecoxib (200 mg); lumiracoxib (100-200 mg); JTE-522; NS-398; and CS-502).

While the experienced clinician is able to monitor and adjust dosages for each patient relative to the severity of pain and the presence of side effects, approximate maximum daily dosages are as follows: flurbiprofen 300 mg; naproxen 1500 mg; naproxen sodium 1650 mg; oxaprozin 1800 mg; etodolac 1200 mg; indomethacin 150-200 mg; ketorolac 120 mg i.m. and 40 mg when taken orally; nabumetone 2000 mg; mefenamic acid 1000 mg; and piroxicam 20 mg. In particular instances, however, exceeding these “maximum” dosages may be the therapeutic choice of a medical professional.

B. 5-HT_{1F} Agonists

As discussed previously the most preferred 5-HT_{1F} agonists are described in international application PCT/US0308455. However there have been many other references in which agonist compounds are described that are suitable for use in the present invention. U.S. Pat. No. 6,380,201 describes one group of compounds that includes optionally substituted compounds of Formula I: 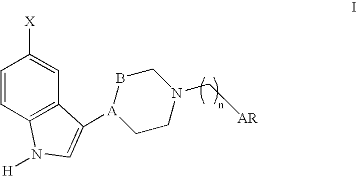
in which
A-B is —CH—CH₂— or —C═CH—;
X is H, halo, C₁-C₄ alkoxy, C₁-C₄ alkylthio, C₁-C₄ alkyl, benzyloxy, hydroxy or carboxamido;
n is 1-4;
Ar is pyridinyl, pyrrolyl or a structure of Formula II: 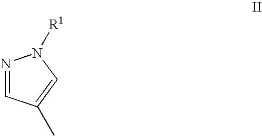
where R¹ is H, C₁-C₆ alkyl, C₃-C₇ cycloalkyl, C₃-C₇ cycloalkylmethyl, benzyl, phenyl or substituted phenyl. These compounds, their synthesis, and use as 5-HT_{1F} agonists are also described in U.S. Pat. No. 5,521,196.

Other 5-HT1F agonists that may be used in the invention are optionally substituted compounds of Formula III: 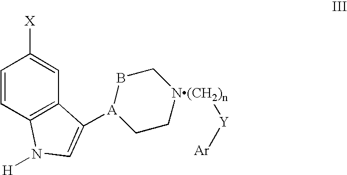
in which
A-B is —CH—CH₂— or —C═CH—;
X is H, halo, C₁-C₄ alkoxy, C₁-C₄ alkylthio, C₁-C₄ alkyl, benzyloxy, hydroxy or carboxamido;
Y is O, S or a bond;
n is 1-4;
Ar is 1-naphthyl, 2-naphtyl, phenyl or phenyl monosubstituted with a substituent selected from the group consisting of halo, C₁-C₄ alkoxy, C₁-C₄ alkylthio, C₁-C₄ alkyl, benzyloxy, hydroxy or trifluoromethyl. These compounds, their synthesis, and use as 5-HT_{1F} agonists are described in U.S. Pat. No. 5,521,197.

Another group of compounds useful for the invention are 5-substituted-3-(1,2,3,6-tetrahydropyridin-4-yl)-1H-indoles and 5-substituted-3-(piperidin-4-yl)-1H-indoles of Formula IV: 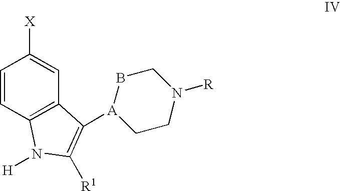
in which
A-B is —CH—CH₂— or —C═CH—;
R is H or C₁-C₆ alkyl;
R¹ is H or C₁-C₄ alkyl;
X is —S—R², —C(O)R³, —C(O)NR⁴R¹⁵, —NR⁵R⁶, —R⁷SO₂R⁸, —NHC(O)NR¹⁰R¹¹, —NH C(O)OR¹² or —NR¹³C(O)R¹⁴;
where
Q is O, or S;
R² is phenyl, substituted phenyl, phenyl(C₁-C₄ alkylene), phenyl(C₁-C₄ alkylene) substituted in the phenyl ring, or pyridinyl;
R³ is C₁-C₆ alkyl, phenyl(C₁-C₄ alkylene), phenyl(C₁-C₄ alkylene) substituted in the phenyl ring, naphthyl, N-methyl-N-methoxyamino, heteroaryl, substituted heteroaryl, heteroaryl(C₁-C₄ alkyl), or substituted heteroaryl(C₁-C₄ alkyl);
R⁴ is heteroaryl, substituted heteroaryl, heteroaryl(C₁-C₄ alkyl), or substituted heteroaryl(C₁-C₄ alkyl);
R⁴ and R¹⁵ taken together with the nitrogen atom form a pyrrolidine, piperidine, substituted piperidine, piperazine, 4-substituted piperazine, morpholine or thiomorpholine ring;
R⁵ and R⁶ are both trifluoromethanesulfonyl;
R⁷ is H or C₁-C₄ alkyl;
R⁸ is C₁-C₄ alkyl, phenyl, substituted phenyl, or di(C₁-C₄ alkyl)amino;
R¹⁰ and R¹¹ are independently selected from the group consisting of C₁-C₆ alkyl, C₃-C₆ alkenyl, C₃-C₈ cycloalkyl, phenyl, substituted phenyl, phenyl(C₁-C₄ alkylene), phenyl (C₁-C₄ alkylene) substituted in the phenyl ring, (C₁-C₄ alkyl or C₁-C₄ alkoxycarbonyl substituted) C₁-C₄ alkyl)phenyl, C₁-C₄ alkyl alpha-substituted with C₁-C₄ alkoxycarbonyl; or
R¹⁰ and R¹¹ taken together with the nitrogen atom form a pyrrolidine, piperidine, piperazine, 4-substituted piperazine, morpholine or thiomorpholine ring;
R¹² is C₁-C₆ alkyl, C₃-C₆ alkenyl, phenyl, substituted phenyl, C₃-C₈ cycloalkyl, C₁-C₄ alkyl ω-substituted with C₁-C₄ alkoxy;
R¹³ is H or C₁-C₄ alkyl;
R¹⁴ is C₁-C₁₀ alkyl substituted with up to three substituents selected from the group consisting of hydroxy, C₁-C₄ alkoxy, halo, aryloxy, C₁-C₄ alkoxycarbonyl and heteroaryloxy, C₂-C₁₀ alkenyl, C₂-C₁₀ alkynyl, C₃-C₈ cycloalkyl, phenyl, substituted phenyl, naphthyl, phenyl(C₁-C₄ alkylene), phenyl(C₁-C₄ alkylene) substituted on the phenyl ring, 2-phenylethylen-1-yl, diphenylmethyl, benzofused C₄-C₈ cycloalkyl, C₁-C₄ alkylene (ω-substituted with C₃-C₆ cycloalkyl, or a heterocycle; and
R¹⁵ is H or C₁-C₆ alkyl.

The chemical terms used in connection with the compounds of formula IV above are defined as having their usual meanings. For example, the terms “alkyl, alkoxy and alkylthio” include such groups as methyl, ethyl, n-propyl, isopropyl, n-butyl, isobutyl, sec-butyl, tert-butyl, pentyl, 2-pentyl-, 3-pentyl-, neopentyl, hexyl, heptyl, octyl and the like. The term “alkenyl” includes vinyl, allyl, 1-buten-4-yl, 2-buten-4-yl, 1-penten-5-yl, 2-penten-5-yl, 3-penten-5-yl, 1-hexen-6-yl, 2-hexen-6-yl, 3-hexen-6-yl, 4-hexen-6-yl and the like. The term “alkynyl” includes acetylenyl, propynyl, 2-butyn-4-yl, 1-butyn-4-yl, 1-pentyn-5-yl, 2-pentyn-5-yl and the like. The term “acyl” includes, for example, formyl, acetyl, propanoyl, butanoyl, and 2-methylpropanoyl. The term “cycloalkyl” includes such groups as cyclopropyl, cyclobutyl, cyclopentyl, cyclohexyl, cycloheptyl and cyclooctyl. The term “phenyl(C₁-C₄ alkylene)” includes such groups as benzyl, phenethyl, phenpropyl and phenbutyl. The term “(C₁-C₄ alkyl)sulfonyl” includes methanesulfonyl, ethanesulfonyl propanesulfonyl, isopropanesulfonyl, butanesulfonyl and the like. The term “halo” includes fluoro, chloro, bromo and iodo.

The term “substituted phenyl” or “phenyl(C₁-C₄ alkylene) substituted in the phenyl ring” is taken to mean the phenyl moiety may be substituted with one substituent selected from the group consisting of halo, C₁-C₄ alkyl, C₁-C₈ alkoxy, C₁-C₄ alkylthio, nitro, cyano, di(C₁-C₄ alkyl)amino, trifluoromethyl, trifluoromethoxy, phenyl, C₁-C₄ acyl, benzoyl or (C₁-C₄ alkyl)sulfonyl, or two to three substituents independently selected from the group consisting of halo, nitro, C₁-C₄ alkyl, or C₁-C₄ alkoxy.

The term “heterocycle” is taken to mean stable aromatic and non-aromatic 5- and 6-membered rings containing from 1 to 3 heteroatoms selected from the group consisting of nitrogen, oxygen and sulfur, said rings being optionally benzofused. All of these rings may be substituted with up to three substituents independently selected from the group consisting of halo, C₁-C₄ alkoxy, C₁-C₄ alkyl, cyano, nitro, hydroxy, —S(O)ₙ—(C₁-C₄ alkyl) and —S(O)ₙ-phenyl where n is 0, 1 or 2. Non-aromatic rings include, for example, pyrrolidinyl, piperidinyl, piperazinyl, tetrahydrofuryl, oxazolidinyl, dioxanyl, pyranyl, and the like. Benzofused non-aromatic rings include indolinyl, 1,2,3,4-tetrahydroquinolinyl, 1,2,3,4-tetrahydroisoquinolinyl and the like. Aromatic rings include furyl, thienyl, pyridinyl, pyrrolyl, N-methylpyrrolyl, oxazolyl, isoxazolyl, pyrazolyl, imidazolyl, triazolyl, oxadiazolyl, thiadiazolyl, thiazolyl, pyrimidinyl, pyrazinyl, pyridazinyl, and the like. Benzofused aromatic rings include isoquinolinyl, benzoxazolyl, benzthiazolyl, quinolinyl, benzofuranyl, thionaphthyl, indolyl and the like.

The term “heteroaryl” is taken to mean an aromatic or benzofused aromatic heterocycle as defined in the previous paragraph. The term “substituted heteroaryl” is taken to mean an aromatic or benzofused aromatic heterocycle as defined in the previous paragraph substituted with up to three substituents independently selected from the group consisting of halo, C₁-C₄ alkoxy, C₁-C₄ alkyl, cyano, nitro, hydroxy, —S(O)ₙ—(C₁-C₄ alkyl) and —S(O)ₙ-phenyl where n is 0, 1 or 2. The term “heteroaryl(C₁-C₄ alkyl) is taken to mean a branched or linear alkyl chain of 1 to 4 carbon atoms substituted at some point with an aromatic or benzofused aromatic heterocycle moiety. The term “substituted heteroaryl(C₁-C₄ alkyl)” is taken to mean a branched or linear alkyl chain of 1 to 4 carbon atoms substituted at some point with an aromatic or benzofused aromatic heterocycle moiety which is substituted with up to three substituents independently selected from the group consisting of halo, C₁-C₄ alkoxy, C₁-C₄ alkyl, cyano, nitro, hydroxy, —S(O)ₙ—C₁-C₄ alkyl) and —S(O)ₙ-phenyl where n is 0, 1 or 2.

The term “heteroaryloxy” is taken to mean a heteroaryl or substituted heteroaryl group, as defined in the previous paragraph, bonded to an oxygen atom.

The term “aryloxy” is taken to mean a phenyl or substituted phenyl group bonded to an oxygen atom.

The term “4-substituted piperazine” is taken to mean a piperazine ring substituted at the 4-position with a substituent selected from the group consisting of C₁-C₆ alkyl, C₁-C₄ alkoxy substituted C₁-C₆ alkyl, phenyl, substituted phenyl, phenyl(C₁-C₄ alkylene), phenyl(C₁-C₄ alkylene) substituted in the phenyl ring, heteroaryl, and heteroaryl C₁-C₄ alkylene).

The term “substituted piperidine” is taken to mean a piperidine ring optionally substituted with a substituent selected from the group consisting of hydroxy, hydroxymethyl, and N,N-di(C₁-C₄ alkyl)carboxamido.

The term “benzofused C₄-C₈ cycloalkyl” is taken to mean a C₄-C₈ cycloalkyl group fused to a phenyl ring. Examples of these groups include benzocyclobutyl, indanyl, 1,2,3,4-tetrahydronaphthyl, and the like.

The compounds of Formula IV may be prepared by methods well known in the art, such as those described in U.S. Pat. No. 4,443,451. Synthetic methods relevant to all of the compounds described above are set forth in U.S. Pat. No. 6,380,201. This patent and all others mentioned herein are hereby incorporated by reference in their entirety.

U.S. Pat. No. 6,777,428 describes methods for making piperidine derivatives that act as 5-HT_{1F} agonists and that can be used in the compositions and methods described herein. Among the appropriate compounds described therein are:
4-[3-((3-trifluoromethylphenyl)sulfonyloxy)benzoyl]-piperidine;
4-[3-((4-trifluoromethoxyphenyl)sulfonyloxy)benzoyl]-1-methylpiperidine;
4-[3-((3-bromophenyl)sulfonyloxy)benzoyl]-1-ethylpiperidine;
4-[3-((3-trifluoromethoxyphenyl)sulfonyloxy)benzoyl]-1-propylpiperidine;
4-[3-((4-chlorophenyl)sulfonyloxy)benzoyl]-1-butylpiperidine;
4-[3-((2-hydroxyphenyl)sulfonyloxy)benzoyl]-1-pentenylpiperidine;
4-[3-((4-bromophenyl)sulfonyloxy)benzoyl]-1-methylpiperidine;
4-[3-((3,5-difluorophenyl)sulfonyloxy)benzoyl]-1-propenylpiperidine;
4-[3-((3-methylphenyl)sulfonyloxy)benzoyl]-1-butenylpiperidine;
4-[3-((pyrid-3-yl)sulfonyloxy)benzoyl]-1-methylpiperidine;
4-[3-((pyrid-2-yl)sulfonyloxy)benzoyl]-1-methylpiperidine;
4-[3-((2,3,4,5,6-pentafluorophenyl)sulfonyloxy)benzoyl]-1-hexenylpiperidine;
4-[3-((4-methylphenyl)sulfonyloxy)benzoyl]-1-propynylpiperidine;
4-[3-((3,4,5-trifluorophenyl)sulfonyloxy)benzoyl]-1-butynylpiperidine;
4-[3-((2,3,4,5-tetrafluorophenyl)sulfonyloxy)benzoyl]-1-pentynylpiperidine;
4-[3-((2-trifluoromethylphenyl)sulfonyloxy)benzoyl]-1-hexynylpiperidine;
4-[3-((4-fluorophenyl)sulfonyloxy)benzoyl]-1-(phenylmethyl)piperidine;
4-[3-((3-chlorophenyl)sulfonyloxy)benzoyl]-1-(phenylethyl)piperidine;
4-[3-((4-iodophenyl)sulfonyloxy)benzoyl]-1-methylpiperidine;
4-[3-((3-fluorophenyl)sulfonyloxy)benzoyl]-1-(2-phenylpropyl)piperidine;
4-[3-((4-methoxyphenyl)sulfonyloxy)benzoyl]-1-(pyrrolidin-2-ylmethyl)piperidine;
4-[3-((2-methylphenyl)sulfonyloxy)benzoyl]-1-(piperidin-1-ylethyl)piperidine;
4-[3-((4-nitrophenyl)sulfonyloxy)benzoyl]-1-methylpiperidine;
4-[3-((2,3-difluorophenyl)sulfonyloxy)benzoyl]-1-(piperazin-2-ylpropyl)piperidine;
4-[3-((fur-2-yl)sulfonyloxy)benzoyl]-1-methylpiperidine;
4-[3-((thiophen-2-yl)sulfonyloxy)benzoyl]-1-methylpiperidine;
4-[3-((2,3,4-trifluorophenyl)sulfonyloxy)benzoyl]-1-(thien-2-ylmethyl)piperidine;
4-[3-((pyridin-4-yl)sulfonyloxy)benzoyl]-1-methylpiperidine;
4-[3-((4-cyanophenyl)sulfonyloxy)benzoyl]-1-methylpiperidine;
4-[3-((3,4-difluorophenyl)sulfonyloxy)benzoyl]-1-(dioxan-2-ylmethyl)piperidine;
4-[3-((2-fluorophenyl)sulfonyloxy)benzoyl]-1-methylpiperidine;
4-[3-((2-trifluoromethoxyphenyl)sulfonyloxy)benzoyl]-1-methylpiperidine;
4-[3-((4-fluorophenyl)sulfonyloxy)benzoyl]-1-methylpiperidine hydrochloride;
4-[3-(phenylsulfonyloxy)benzoyl]-1-methylpiperidine;
4-[3-((2-bromophenyl)sulfonyloxy)benzoyl]-1-methylpiperidine;
4-[3-((2,3,5-trifluorophenyl)sulfonyloxy)benzoyl]-1-methylpiperidine hydrobromide;
4-[3-((2-nitrophenyl)sulfonyloxy)benzoyl]-1-methylpiperidine;
4-[3-((2,4,5-trifluorophenyl)sulfonyloxy)benzoyl]-1-methylpiperidine sulfide;
4-[3-((2-iodophenyl)sulfonyloxy)benzoyl]-1-methylpiperidine;
4-[3-(3-nitrophenylthioureido)benzoyl]-1-methylpiperidine;
4-[3-(3-trifluoromethylphenylthioureido)benzoyl]-1-methylpiperidine oxalate;
4-[3-(4-trifluoromethoxyphenylthioureido)benzoyl]-1-methylpiperidine methanesulfonate;
4-[3-(phenylthioureido)benzoyl]-1-methylpiperidine;
4-[3-(3-bromophenylthioureido)benzoyl]-1-methylpiperidine;
4-[3-(3-trifluoromethoxyphenylthioureido)benzoyl]-1-methylpiperidine fumarate;
4-[3-(4-chlorophenylthioureido)benzoyl]-1-methylpiperidine;
4-[3-(2-hydroxyphenylthioureido)benzoyl]-1-methylpiperidine;
4-[3-(4-bromophenylthioureido)benzoyl]-1-methylpiperidine;
4-[3-(3,5-difluorophenylthioureido)benzoyl]-1-methylpiperidine phthalate;
4-[3-(3-methylphenylthioureido)benzoyl]-1-methylpiperidine;
4-[3-(pyrid-3-ylthioureido)benzoyl]-1-methylpiperidine;
4-[3-(pyrid-2-ylthioureido)benzoyl]-1-methylpiperidine;
4-[3-(2,3,4,5,6-pentafluorophenylthioureido)benzoyl]-1-methylpiperidine chlorobenzoate;
4-[3-(4-methylphenylthioureido)benzoyl]-1-methylpiperidine;
4-[3-(3,4,5-trifluorophenylthioureido)benzoyl]-1-methylpiperidine citrate;
4-[3-(2,3,4,5-tetrafluorophenylthioureido)benzoyl]-1-methylpiperidine tartrate;
4-[3-(2-trifluoromethylphenylthioureido)benzoyl]-1-methylpiperidine propanesulfonate;
4-[3-(4-fluorophenylthioureido)benzoyl]-1-methylpiperidine;
4-[3-(3-chlorophenylthioureido)benzoyl]-1-methylpiperidine;
4-[3-(4-iodophenylthioureido)benzoyl]-1-methylpiperidine;
4-[3-(3-fluorophenylthioureido)benzoyl]-1-methylpiperidine;
4-[3-(4-methoxyphenylthioureido)benzoyl]-1-methylpiperidine;
4-[3-(2-methylphenylthioureido)benzoyl]-1-methylpiperidine;
4-[3-(4-nitrophenylthioureido)benzoyl]-1-methylpiperidine;
4-[3-(2,3-difluorophenylthioureido)benzoyl]-1-methylpiperidine hydroxybenzoate;
4-[3-(fur-2-ylthioureido)benzoyl]-1-methylpiperidine;
4-[3-(thiophen-2-ylthioureido)benzoyl]-1-methylpiperidine;
4-[3-(2,3,4-trifluorophenylthioureido)benzoyl]-1-methylpiperidine decanoate;
4-[3-(pyridin-4-ylthioureido)benzoyl]-1-methylpiperidine;
4-[3-(4-cyanophenylthioureido)benzoyl]-1-methylpiperidine;
4-[3-(3,4-difluorophenylthioureido)benzoyl]-1-methylpiperidine monohydrogenphosphate;
4-[3-(2-fluorophenylthioureido)benzoyl]-1-methylpiperidine;
4-[3-(2-trifluoromethoxyphenylthioureido)benzoyl]-1-1-methylpiperidine sulfite;
4-[3-(4-fluorophenylthioureido)benzoyl]-1-methylpiperidine;
4-[3-(2-bromophenylthioureido)benzoyl]-1-methylpiperidine;
4-[3-(2,3,5-trifluorophenylthioureido)benzoyl]-1-methylpiperidine pyrosulfate;
4-[3-(2-nitrophenylthioureido)benzoyl]-1-methylpiperidine;
4-[3-(2,4,5-trifluorophenylthioureido)benzoyl]-1-ethylpiperidine malonate;
4-[3-(2-iodophenylthioureido)benzoyl]-1-methylpiperidine;
4-[3-(3-nitrophenylureido)benzoyl]-1-methylpiperidine;
4-[3-(3-trifluoromethylphenylureido)benzoyl]-1-methylpiperidine xylenesulfonate;
4-[3-(4-trifluoromethoxyphenylureido)benzoyl]-1-propylpiperidine glycollate;
4-[3-(phenylureido)benzoyl]-1-methylpiperidine;
4-[3-((+)-2-hydroxypropylureido)benzoyl]-1-methylpiperidine;
4-[3-((−)-3-phenylbutylureido)benzoyl]-1-methylpiperidine;
4-[3-(R-2-(diphenylmethyl)propylureido)benzoyl]-1-methylpiperidine;
4-[3-(S-2-hydroxypropylureido)benzoyl]-1-methyl-piperidine;
4-[3-(3-trifluoromethoxyphenylureido)benzoyl]-1-methylpiperidine lactate;
4-[3-(4-chlorophenylureido)benzoyl]-1-methylpiperidine;
4-[3-(2-hydroxyphenylureido)benzoyl]-1-methylpiperidine;
4-[3-(4-bromophenylureido)benzoyl]-1-methylpiperidine;
4-[3-(3,5-difluorophenylureido)benzoyl]-1-methylpiperidine;
4-[3-(3-methylphenylureido)benzoyl]-1-methylpiperidine;
4-[3-(pyrid-3-ylureido)benzoyl]-1-methylpiperidine;
4-[3-(pyrid-2-ylureido)benzoyl]-1-methylpiperidine;
4-[3-(2,3,4,5,6-pentafluorophenylureido)benzoyl]-1-methylpiperidine mandelate;
4-[3-(4-methylphenylureido)benzoyl]-1-methylpiperidine;
4-[3-(3,4,5-trifluorophenylureido)benzoyl]-1-methylpiperidine lactate;
4-[3-(2,3,4,5-tetrafluorophenylureido)benzoyl]-1-methylpiperidine caprylate;
4-[3-(2-trifluoromethylphenylureido)benzoyl]-1-methylpiperidine acrylate;
4-[3-(4-fluorophenylureido)benzoyl]-1-methylpiperidine;
4-[3-(3-chlorophenylureido)benzoyl]-1-methylpiperidine;
4-[3-(4-iodophenylureido)benzoyl]-1-methylpiperidine;
4-[3-(3-fluorophenylureido)benzoyl]-1-methylpiperidine;
4-[3-(4-methoxyphenylureido)benzoyl]-1-methylpiperidine;
4-[3-(2-methylphenylureido)benzoyl]-1-methylpiperidine;
4-[3-(4-nitrophenylureido)benzoyl]-1-methylpiperidine;
4-[3-(2,3-difluorophenylureido)benzoyl]-1-methylpiperidine;
4-[3-(fur-2-ylureido)benzoyl]-1-methylpiperidine;
4-[3-(thiophen-2-ylureido)benzoyl]-1-methylpiperidine;
4-[3-(2,3,4-trifluorophenylureido)benzoyl]-1-methylpiperidine formate;
4-[3-(pyridin-4-ylureido)benzoyl]-1-methylpiperidine;
4-[3-(4-cyanophenylureido)benzoyl]-1-methylpiperidine;
4-[3-(3,4-difluorophenylureido)benzoyl]-1-methylpiperidine;
4-[3-(2-fluorophenylureido)benzoyl]-1-methylpiperidine;
4-[3-(2-trifluoromethoxyphenylureido)benzoyl]-1-methylpiperidine iodide;
4-[3-(4-fluorophenylureido)benzoyl]-1-methylpiperidine;
4-[3-(2-bromophenylureido)benzoyl]-1-methylpiperidine;
4-[3-(2,3,5-trifluorophenylureido)benzoyl]-1-methylpiperidine;
4-[3-(2-nitrophenylureido)benzoyl]-1-methylpiperidine;
4-[3-(2,4,5-trifluorophenylureido)benzoyl]-1-methylpiperidine;
4-[3-(2-iodophenylureido)benzoyl]-1-methylpiperidine;
4-[3-(3-nitrophenylsulfonamino)benzoyl]-1-methylpiperidine;
4-[3-(3-trifluoromethylphenylsulfonamino)benzoyl]-1-methylpiperidine;
4-[3-(4-trifluoromethoxyphenylsulfonamino)benzoyl]-1-methylpiperidine;
4-[3-phenylsulfonamino)benzoyl]-1-methylpiperidine;
4-[3-(3-bromophenylsulfonamino)benzoyl]-1-methylpiperidine;
4-[3-(3-trifluoromethoxyphenylsulfonamino)benzoyl]-1-methylpiperidine;
4-[3-(4-chloro phenylsulfonamino)benzoyl]-1-methylpiperidine;
4-[3-(2-hydroxyphenylsulfonamino)benzoyl]-1-methylpiperidine;
4-[3-(4-bromophenylsulfonamino)benzoyl]-1-methylpiperidine;
4-[3-(3,5-difluorophenylsulfonamino)benzoyl]-1-methylpiperidine;
4-[3-(3-methylphenylsulfonamino)benzoyl]-1-methylpiperidine;
4-[3-(pyrid-3-ylsulfonamino)benzoyl]-1-methylpiperidine;
4-[3-(pyrid-2-ylsulfonamino)benzoyl]-1-methylpiperidine;
4-[3-(2,3,4,5,6-pentafluorophenylsulfonamino)benzoyl]-1-methylpiperidine;
4-[3-(4-methylphenylsulfonamino)benzoyl]-1-methylpiperidine;
4-[3-(3,4,5-trifluorophenylsulfonamino)benzoyl]-1-methylpiperidine;
4-[3-(2,3,4,5-tetrafluorophenylsulfonamino)benzoyl]-1-methylpiperidine;
4-[3-(2-trifluoromethylphenylsulfonamino)benzoyl]-1-methylpiperidine;
4-[3-(4-fluorophenylsulfonamino)benzoyl]-1-methylpiperidine;
4-[3-(3-chlorophenylsulfonamino)benzoyl]-1-methylpiperidine;
4-[3-(4-iodophenylsulfonamino)benzoyl]-1-methylpiperidine;
4-[3-(3-fluorophenylsulfonamino)benzoyl]-1-methylpiperidine;
4-[3-(4-methoxyphenylsulfonamino)benzoyl]-1-methylpiperidine;
4-[3-(2-methylphenylsulfonamino)benzoyl]-1-methylpiperidine;
4-[3-(4-nitrophenylsulfonamino)benzoyl]-1-methylpiperidine;
4-[3-(2,3-difluorophenylsulfonamino)benzoyl]-1-methylpiperidine;
4-[3-(fur-2-ylsulfonamino)benzoyl]-1-methylpiperidine;
4-[3-(thiophen-2-ylsulfonamino)benzoyl]-1-methylpiperidine;
4-[3-(2,3,4-trifluorophenylsulfonamino)benzoyl]-1-methylpiperidine;
4-[3-(pyridin-4-ylsulfonamino)benzoyl]-1-methylpiperidine;
4-[3-(4-cyanophenylsulfonamino)benzoyl]-1-methylpiperidine;
4-[3-(3,4-difluorophenylsulfonamino)benzoyl]-1-methylpiperidine;
4-[3-(2-fluorophenylsulfonamino)benzoyl]-1-methylpiperidine;
4-[3-(2-trifluoromethoxyphenylsulfonamino)benzoyl]-1-methylpiperidine;
4-[3-(4-fluorophenylsulfonamino)benzoyl]-1-methylpiperidine;
4-[3-(2-bromophenylsulfonamino)benzoyl]-1-methylpiperidine;
4-[3-(2,3,5-trifluorophenylsulfonamino)benzoyl]-1-methylpiperidine;
4-[3-(2-nitrophenylsulfonamino)benzoyl]-1-methylpiperidine;
4-[3-(2,4,5-trifluorophenylsulfonamino)benzoyl]-1-methylpiperidine;
4-[3-(2-iodophenylsulfonamino)benzoyl]-1-methylpiperidine;
4-[3-((3-trifluoromethylphenyl)amidyl)benzoyl]-1-methylpiperidine;
4-[3-((4-trifluoromethoxyphenyl)amidyl)benzoyl]-1-methylpiperidine;
4-[3-((3-bromophenyl)amidyl)benzoyl]-1-methylpiperidine;
4-[3-((3-trifluoromethoxyphenyl)amidyl)benzoyl]-1-methylpiperidine;
4-[3-((4-chlorophenyl)amidyl)benzoyl]-1-methylpiperidine;
4-[3-((2-hydroxyphenyl)amidyl)benzoyl]-1-methylpiperidine;
4-[3-((4-bromophenyl)amidyl)benzoyl]-1-methylpiperidine;
4-[3-((3,5-difluorophenyl)amidyl)benzoyl]-1-methylpiperidine;
4-[3-((3-methylphenyl)amidyl)benzoyl]-1-methylpiperidine;
4-[3-((pyrid-3-yl)amidyl)benzoyl]-1-methylpiperidine;
4-[3-((pyrid-2-yl)amidyl)benzoyl]-1-methylpiperidine;
4-[3-((2,3,4,5,6-pentafluorophenyl)amidyl)benzoyl]-1-methylpiperidine;
4-[3-((4-methylphenyl)amidyl)benzoyl]-1-methylpiperidine;
4-[3-((3,4,5-trifluorophenyl)amidyl)benzoyl]-1-methylpiperidine;
4-[3-((2,3,4,5-tetrafluorophenyl)amidyl)benzoyl]-1-methylpiperidine;
4-[3-((2-trifluoromethylphenyl)amidyl)benzoyl]-1-methylpiperidine;
4-[3-((4-fluorophenyl)amidyl)benzoyl]-1-methylpiperidine;
4-[3-((3-chlorophenyl)amidyl)benzoyl]-1-methylpiperidine;
4-[3-((4-iodophenyl)amidyl)benzoyl]-1-methylpiperidine;
4-[3-((3-fluorophenyl)amidyl)benzoyl]-1-methylpiperidine;
4-[3-((4-methoxyphenyl)amidyl)benzoyl]-1-methylpiperidine;
4-[3-((2-methylphenyl)amidyl)benzoyl]-1-methylpiperidine;
4-[3-((4-nitrophenyl)amidyl)benzoyl]-1-methylpiperidine;
4-[3-((2,3-difluorophenyl)amidyl)benzoyl]-1-methylpiperidine;
4-[3-((fur-2-yl)amidyl)benzoyl]-1-methylpiperidine;
4-[3-((thiophen-2-yl)amidyl)benzoyl]-1-methylpiperidine;
4-[3-((2,3,4-trifluorophenyl)amidyl)benzoyl]-1-methylpiperidine;
4-[3-((pyridin-4-yl)amidyl)benzoyl]-1-methylpiperidine;
4-[3-((4-cyanophenyl)amidyl)benzoyl]-1-methylpiperidine;
4-[3-((3,4-difluorophenyl)amidyl)benzoyl]-1-methylpiperidine;
4-[3-((2-fluorophenyl)amidyl)benzoyl]-1-methylpiperidine;
4-[3-((2-trifluoromethoxyphenyl)amidyl)benzoyl]-1-methylpiperidine;
4-[3-((4-fluorophenyl)amidyl)benzoyl]-1-methylpiperidine;
4-[3-(phenylamidyl)benzoyl]-1-methylpiperidine;
4-[3-((2-bromophenyl)amidyl)benzoyl]-1-methylpiperidine;
4-[3-((2,3,5-trifluorophenyl)amidyl)benzoyl]-1-methylpiperidine;
4-[3-((2-nitrophenyl)amidyl)benzoyl]-1-methylpiperidine;
4-[3-((2,4,5-trifluorophenyl)amidyl)benzoyl]-1-methylpiperidine.

Still other 5-HT1F agonists compatible with the invention are the 5-substituted-3-(1,2,3,6-tetrahydropyridin-4-yl)- and 3-(piperidin-4-yl)-1H-indoles described in U.S. Pat. No. 5,962,474 and U.S. Pat. No. 5,708,008 and the N-2-substituted-3-(2-aminoethyl)-1H-indol-5-yl-amides described in U.S. Pat. No. 5,942,536.

U.S. Pat. Nos. 6,696,439 and 6,133,290 describe the structure and synthesis of additional 5-HT1F agonists that my be used in conjunction with the present invention. Agonists described in the '439 patent include:
2-methyl-3-(2-[N′,N′-diethylamino]ethyl)-5-(4-propanesulfonylbenzamide)furo[3,2-]pyridine hydrochloride;
2-n-butyl-3-(2-[N′-methyl-N′-benzylamino]ethyl)-5-(4-fluorobenzamide)furo[3,2-b]pyridine;
2-isobutyl-3-(2-[N′-methyl-N′-cyclopropylmethylamino]ethyl)-5-(4-iodobenzamide)furo [3,2-b]pyridine naphthalene-1-sulfonate;
2-s-butyl-3-(2-[N′-methyl-N′-(2-[1-propylpyrazol-4-yl]ethyl)amino]ethyl)-5-(4-fluorobenz-amide)furo[3,2-b]pyridine ditoluoyltartrate;
2-methyl-3-(2-[N′-methyl-N′-s-butylaminoethyl)-5-isobutyramidefuro[3,2-b]pyridine;
2-methyl-3-(2-[N′-methyl-N′-(2-[pyridin-4-yl]ethyl)amino]ethyl)-5-(4-fluorobenzamide) furo[3,2-b]pyridine malonate;
2-methyl-3-(2-[N′-methyl-N′-(2-[1-isbpropylpyrazol-4-yl]ethyl)amino]ethyl)-5-butyramide furo[3,2-b]pyridine mandelate;
3-(2-[N′-methyl-N′-([4-bromothien-2-yl]methyl)amino]ethyl)-5-(4-fluorobenzamide)furo [3,2-b]pyridine hydrochloride;
2-ethyl-3-(2-[N′-ethyl-N′-(2-[3-methylthiobenzofur-5-yl]ethyl)amino]ethyl)-5-(pyridine-2-carboxamide)furo[3,2-b]pyridine;
2-propyl-3-(2-[N′-isopropyl-N′-(3-[isobenzofur-2-yl]propyl)amino]ethyl)-5-(4-fluorobenz-amide)furo[3,2-b]pyridine;
2-methyl-3-(2-[N′-butyl-N′-([pyrrol-3-yl]methyl)amino]ethyl)-5-(4-fluorobenzamide)furo [3,2-b]pyridine maleate;
2-methyl-3-(2-[N′-methyl-N′-([5-cyanoimidazol-2-yl]methyl)amino]ethyl)-5-acetamidefuro [3,2-b]pyridine trifluoroacetate;
2-methyl-3-(2-[N′-methyl-N′-([6-carboxamidopyrazin-2-yl]methyl)amino]ethyl)-5-propane carboxamidefuro[3,2-b]pyridine;
2-methyl-3-(2-[N′-methyl-N′-([5-nitropyrimidin-2-yl]methyl)amino]ethyl)-5-(2-propane carboxamide)furo[3,2-b]pyridine;
2-methyl-3-(2-[N′-methyl-N′-([5-dimethylaminopyridazin-3-yl]methyl)amino]ethyl)-5-butyramidefuro[3,2-b]pyridine benzoate;
2-methyl-3-(2-[N′-methyl-N′-([indazol-5-yl]methyl)amino]ethyl)-5-pentanecarboxamide furo[3,2-b]pyridine;
2-methyl-3-(2-[N′-methyl-N′-([quinolin-4-yl]methyl)amino]ethyl)-5-cyclopropanecarbox amide furo[3,2-b]pyridine;
2-methyl-3-(2-[N′-methyl-N′-([isoquinolin-7-yl]methyl)amino]ethyl)-5-cyclobutanecarbox amidefuro[3,2-b]pyridine;
2-methyl-3-(2-[N′-methyl-N′-([quinoxalin-2-yl]methyl)amino]ethyl)-5-cyclopentanecarbox amidefuro[3,2-b]pyridine hexanoate;
2-methyl-3-(2-[N′-methyl-N′-([quinaxolin-5-yl]methyl)amino]ethyl)-5-cyclohexanecarbox amidefuro[3,2-b]pyridine;
2-methyl-3-(2-[N′-methyl-N′-([thiazol-2-yl]methyl)amino]ethyl)-5-cycloheptanecarbox amidefuro[3,2-b]pyridine;
2-methyl-3-(2-[N′-methyl-N′-([2-aminobenzothiazol-5-yl]methyl)amino]ethyl)-5-(4-fluoro benzamide)furo[3,2-b]pyridine trifluoromethanesulfonate;
2-methyl-3-(2-[N′-methyl-N′-([oxazol-5-yl]methyl)amino]ethyl)-5-(3-iodobenzamide)furo [3,2-b]pyridine;
2-methyl-3-(2-[N′-methyl-N′-([6-nitrobenzoxazol-2-yl]methyl)amino]ethyl)-5-(2-chloro benzamide)furo[3,2-b]pyridine hydrobromide;
2-methyl-3-(2-[N′-methyl-N′-([1,4-benzodioxan-6-yl]methyl)amino]ethyl)-5-(2-chloropyri-dine-3-carboxamide)furo[3,2-b]pyridine;
2-isopropyl-3-(2-[N′-methyl-N′-([isoxazol-4-yl]methyl)amino]ethyl)-5-benzamidefuro[3,2-b]pyridine;
2-methyl-3-(2-[N′-methyl-N′-([benzisoxazol-3-yl]methyl)amino]ethyl)-5-(thiophene-2-car boxamide)furo[3,2-b]pyridine;
2-methyl-3-(2-[N′-methyl-N′-([1,3,4-oxadiazol-2-yl]methyl)amino]ethyl)-5-(furyl-3-carbox amide)furo[3,2-b]pyridine;
2-methyl-3-(2-[N′-methyl-N′-([1,2,3-triazol-4-yl]methyl)amino]ethyl)-5-(4-fluorobenz amide) furo[3,2-b]pyridine tosylate;
3-(2-[N′-methyl-N′-((4-bromothien-2-yl)methyl)amino]ethyl)-5-(4-fluorobenzamide)furo [3,2-b]pyridine hydrochloride;
2-ethyl-3-(2-[N′-ethyl-N′-((3-methylthiobenzofur-5-yl)ethyl)amino]ethyl)-5-(pyridine-2-carboxamide)furo[3,2-b]pyridine;
2-propyl-3-(2-[N′-isopropyl-N′-1-((isobenzofur-2-yl)prop-3-yl)amino]ethyl)-5-(4-fluoro benzamide)furo[3,2-b]pyridine;
2-methyl-3-(2-[N′-butyl-N′-(pyrrol-3-yl)methyl)amino]ethyl)-5-(4-fluorobenzamide)furo [3,2-b]pyridine maleate;
2-methyl-3-(2-[N′-methyl-N′-((5-cyanoimidazol-2-yl)methyl)amino]ethyl)-5-(4-acetamide) furo[3,2-b]pyridine trifluoroacetate;
2-methyl-3-(2-[N′-methyl-N′-((6-carboxamidopyrazin-2-yl)methyl)amino]ethyl)-5-propane carboxamidefuro[3,2-b]pyridine;
2-methyl-3-(2-[N′-methyl-N′-((5-nitropyrimidin-2-yl)methyl)amino]ethyl)-5-(2-propanecar boxamide)furo[3,2-b]pyridine;
2-methyl-3-(2-[N′-methyl-N′-((5-dimethylaminopyridazin-3-yl)methyl)amino]ethyl)-5-butanecarboxamidefuro[3,2-b]pyridine benzoate;
2-methyl-3-(2-[N′-methyl-N′-((indazol-5-yl)methyl)amino]ethyl)-5-pentanecarboxamide furo[3,2-b]pyridine;
2-methyl-3-(2-[N′-methyl-N′-((2-aminobenzothiazol-5-yl)methyl)amino]ethyl)-5-(4-fluoro benzamide)furo[3,2-b]pyridine trifluoromethanesulfonate;
2-methyl-3-(2-[N′-methyl-N′-(2-[pyridin-4-yl]ethyl)amino]ethyl)-5-(N-ethylurea)furo[3,2-b]pyridine;
2-methyl-3-(2-[N′-methyl-N′-s-butylamino]ethyl)-5-(N-isopropylurea)furo[3,2-b]pyridine;
2-methyl-3-(2-[N′-methyl-N′-(2-[pyridin-4-yl]ethyl)amino]ethyl)-5-[N-[(3-methoxy)phenyl]urea]furo[3,2-b]pyridine malonate.

Agonists described in the '290 patent include:
5-(N-sec-butanesulfonyl)amino-3-(1-methylpiperidin-4-yl)-1H-indazole;
5-(N,N-dibutylaminosulfonyl)amino-3-(1-azabicyclo[5.4.0]undec-3-en-4-yl)-1H-indazole;
5-(N-isopropyl-N-sec-butanesulfonyl)amino-3-(1-ethylpiperidin-4-yl)-1H-indazole;
N-ethyl-N′-(3-(1,2,3,4,5,8-hexahydroindolizin-7-yl)-1H-indazol-5-yl)thiourea;
N-(3-propoxy)phenyl-N′-(3-(1-propylpiperidin-4-yl)-1H-indazol-5-yl)thiourea;
N-(3,4-difluoro)phenyl-N′-(3-(1,2,3,4,5,8-hexahydroindolizin-7-yl)-1H-indazol-5-yl)thiourea;
N-(3-fluoro-5-chloro)phenyl-N′-(3-(1-butylpiperidin-4-yl)-1H-indazol-5-yl)thiourea;
N-butyl-N′-(3-(octahydroindolizin-7-yl)-1H-indazol-5-yl)thiourea;
N-(2-butoxy)phenyl-N′-(3-(1-pentylpiperidin-4-yl)-1H-indazol-5-yl)thiourea hydrochloride;
N-(2-bromo-4-fluoro)phenyl-N′-(3-(1-hexylpiperidin-4-yl)-1H-indazol-5-yl)thiourea;
N-(4-trifluoromethyl)phenyl-N′-(3-(octahydro-2H-quinolizin-2-yl)-1H-indazol-5-yl)thio urea;
N-(2-hexen-6-yl)-N′-(3-(1-(2-pentyl)-1,2,3,4-tetrahydropyridin-4-yl)-1H-indazol-5-yl)urea;
N-(4-propylthio)phenyl-N′-(3-(1-methylpiperidin-4-yl)-1H-indazol-5-yl)urea;
N-(4-phenbutyl)-N′-(3-(1,4,5,6,7,8,9-heptahydroquinolizin-2-yl)-1H-indazol-5-yl)urea;
N-hexyl-N′-(3-(1-ethylpiperidin-4-yl)-1H-indazol-5-yl)urea;
N-(2-chloro)phenyl-N′-(3-(1-azabicyclo[5.4.0]undecan-4-yl)-1H-indazol-5-yl)urea;
N-(2-isopropoxy)phenyl-N′-(3-(1-propylpiperidin-4-yl)-1H-indazol-5-yl)urea;
N-(2-ethylthio)phenyl-N′-(3-(octahydroindolizin-7-yl)-1H-indazol-5-yl)urea;
N-(2-butyl)phenyl-N′-(3-(1-butylpiperidin-4-yl)-1H-indazol-5-yl)urea;
N-(3,4-difluoro)phenyl-N′-(3-(octahydro-2H-quinolizin-2-yl)-1H-indazol-5-yl)urea;
N-ethyl-N-phenyl-N′-(3-(1-cyclobutylpiperidin-4-yl)-1H-indazol-5-yl)urea;
5-(2-chlorophenoxy)carbonylamino-3-(1,4,5,6,7,8,9-heptahydroquinolizin-2-yl)-1H-indazole;
5-(3-propoxyphenoxy)carbonylamino-3-(1-cyclopentylpiperidin-4-yl)-1H-indazole;
5-(2-buten-4-yloxy)carbonylamino-3-(octahydro-2H-quinolizin-2-yl)-1H-indazole;
5-(2-iodophenoxy)carbonylamino-3-(1-pentyl-4-piperidin-4-yl)-1H-indazole;
5-cyclopropoxycarbonylamino-3-(1-azabicyclo[5.4.0]undecan-4-yl)-1H-indazole;
5-(acetyl)amino-3-(1-cyclohexylpiperidin-4-yl)-1H-indazole;
5-(4-phenoxybutanoyl)amino-3-(1,4,5,6,7,8,9-heptahydroquinolizin-2-yl)-1H-indazole;
5-benzoylamino-3-(1-hexylpiperidin-4-yl)-1H-indazole;
5-(4-fluorobenzoyl)amino-3-(1,4,5,6,7,8,9-heptahydroquinolizin-2-yl)-1H-indazole;
5-(4-fluorobenzoyl)amino-3-(1-methylpiperidin-4-yl)-1H-indazole;
5-(2-chlorobenzoyl)-N-ethylamino-3-(1,4,5,6,7,8,9-heptahydroquinolizin-2-yl)-1H-indazole;
5-(2-propylbenzoyl)amino-3-(1-ethylpiperidin-4-yl)-1H-indazole;
5-(3-heptyloxybenzoyl)amino-3-(1,4,5,6,7,8,9-heptahydroquinolizin-2-yl)-1H-indazole;
5-(2-cyanobenzoyl)amino-3-(1-propylpiperidin-4-yl)-1H-indazole;
5-(4-(propanoyl)benzoyl)amino-3-(1,4,5,6,7,8,9-heptahydroquinolizin-2-yl)-1H-indazole;
5-(2-bromo-3-iodo)benzoylamino-3-(1-cyclopropylpiperidin-4-yl)-1H-indazole;
5-(2-thienoyl)amino-3-(1,4,5,6,7,8,9-heptahydroquinolizin-2-yl)-1H-indazole;
5-(2-thienoyl)amino-3-(1-butylpiperidin-4-yl)-1H-indazole;
5-(3-thienoyl)amino-3-(1,4,5,6,7,8,9-heptahydroquinolizin-2-yl)-1H-indazole;
5-(2-furoyl)amino-3-(1-cyclobutylpiperidin-4-yl)-1H-indazole;
5-(3-furoyl)amino-3-(1,2,3,4,5,8-hexahydroindolizin-7-yl)-1H-indazole;
5-(3-furoyl)amino-3-(1-pentylpiperidin-4-yl)-1H-indazole;
5-(4-phenylbutanoyl)amino-3-(octahydro-2H-quinolizin-2-yl)-1H-indazole;
5-benzoylamino-3-(1-cyclopentylpiperidin-4-yl)-1H-indazole;
5-benzoylamino-3-(octahydroindolizin-7-yl)-1H-indazole;
5-(4-fluorobenzoyl)amino-3-(1-hexylpiperidin-4-yl)-1H-indazole;
5-(2-chlorobenzoyl)amino-3-(octahydroindolizin-7-yl)-1H-indazole;
5-(2-ethylbenzoyl)amino-3-(1-cyclohexylpiperidin-4-yl)-1H-indazole;
5-(2-hexyloxybenzoyl)amino-3-(octahydroindolizin-7-yl)-1H-indazole;
5-(4-(diethylamino)benzoyl)amino-3-(1-methylpiperidin-4-yl)-1H-indazole;
5-(2-(benzoyl)benzoyl)amino-3-(octahydroindolizin-7-yl)-1H-indazole;
5-(2-bromo-4-fluoro)benzoylamino-(3-(1-ethylpiperidin-4-yl)-1H-indazole;
5-(2-thienoyl)amino-3-(octahydroindolizin-7-yl)-1H-indazole;
5-(3-thienoyl)amino-3-(1-propylpiperidin-4-yl)-1H-indazole;
5-(3-thienoyl)amino-3-(octahydroindolizin-7-yl)-1H-indazole;
5-(2-furoyl)amino-3-(1-cyclopropylpiperidin-4-yl)-1H-indazole;
5-(3-furoyl)amino-3-(octahydro-2H-quinolizin-2-yl)-1H-indazole.
   III. Making of Pharmaceutical Compositions

Pharmaceutical compositions and dosage forms can be produced in accordance with conventional methods that are standard in the art (see, e.g., Remington's Pharmaceutical Sciences, 16^{th} ed., A. Oslo. editor, Easton, Pa. (1980)). Active ingredients, individually or in combination, may be mixed with pharmaceutically acceptable organic or inorganic carrier substances suitable for parenteral, enteral (e.g., oral or intranasal) or topical application. Pharmaceutically acceptable carriers include, but are not limited to: water; salt solutions; alcohols; gum arabic; vegetable oils; benzyl alcohols; polyethylene glycols; gelatin; carbohydrates, such as lactose, amylase, or starch; magnesium stearate; talc; titanium dioxide; silicic acid; viscous paraffin; perfume oil; fatty acid esters; etc.

The pharmaceutical preparations can be sterilized and, if desired, mixed with auxiliary agents, e.g., lubricants, preservatives, stabilizers, wetting agents, emulsifiers, salts for controlling osmotic pressure, buffers, coloring agents, flavoring agents, and/or aromatic substances. Compositions can also include other active agents.

If desired, enteric coating layers may be incorporated into tablets or capsules. Coating materials may include one or more of the following: methacrylic acid copolymers, shellac, hydroxypropylmethylcellulose phthalate, polyvinyl acetate phthalate, hydroxy-propylmethylcellulose trimellitate, carboxymethylethylcellulose, cellulose acetate phthalate, or other suitable enteric coating polymers. The pH at which the enteric coat will dissolve can be controlled by the polymer or combination of polymers selected and/or ratio of pendant groups. For example, dissolution characteristics may be affected by the ratio of free carboxyl groups to ester groups. Enteric coating layers may also contain pharmaceutically acceptable plasticizers such as triethyl citrate, dibutyl phthalate, tricetin, polyethylene glycols, polysorbates, etc. Additives, such as dispersants, colorants, anti-adhering and anti-foaming agents, may also be included.

IV. Making of Tablet Dosage Forms

Although the present invention is compatible with any route of administration, oral dosage forms are preferred, with the most preferred being tablets or capsules. Methods for making multilayer tablets are well known in the art and procedures for making coordinated dosage forms, particularly with compositions containing metoclopramide have been previously described (see U.S. Pat. No. 6,479,551). In a multilayer configuration, one portion of a tablet or capsule will contain NSAID, anti-inflammatory agent, or gastric prokinetic agent. Other portions will contain the other drugs along with appropriate excipients, dissolution agents, lubricants, fillers, etc. Tablets may be granulated by methods such as slugging, low- or high-sheer granulation, wet granulation, or fluidized-bed granulation. Of these processes, slugging generally produces tablets of less hardness and greater friability. Low-sheer granulation, high-sheer granulation, wet granulation and fluidized bed granulation generally produce harder, less friable tablets.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A pharmaceutical composition in unit dose form, comprising:
a) a 5-HT_{1F}-specific agonist;
b) an NSAID;
wherein said 5-HT_{1F}-specific agonist and said NSAID are present in an amount effective to relieve pain upon the administration of one or more unit doses to a patient.

2. The pharmaceutical composition of claim 1 wherein said NSAID has an onset of action of two hours or less.

3. The pharmaceutical composition of claim 1 wherein said 5-HT_{1F}-specific agonist and said NSAID are present in an amount sufficient to provide therapeutic synergy or migraine-related therapeutic synergy and wherein neither drug is present in an amount sufficient to provide a therapeutic effect in the absence of the other.

4. The pharmaceutical composition of claim 1, wherein said 5-HT_{1F}-specific agonist is present in said unit dosage form at between 0.5 and 600 mg.

5. The pharmaceutical composition of claim 4, wherein said NSAID is present in said unit dosage form at 1-600 mg.

6. The pharmaceutical composition of claim 4, wherein said 5-HT_{1F}-specific agonist is 2,4,6-trifluoro-N-[6-(1-methyl-piperidin-4-ylcarbonyl)-pyridin-2-yl]-benzamide.

7. The pharmaceutical composition of claim 6, wherein said NSAID is present at 1-600 mg and is selected from the group consisting of: aspirin; ibuprofen; acetaminophen; naproxen; diclofenac; ketorolac; etodolac; flurbiprofen; oxaprozin; indomethacin; mefenamic acid; nabumetone; piroxicam; celecoxib; rofecoxib; valdecoxib; and lornoxicam.

8. A method of treating a patient for migraine or headache pain comprising administering to said patient a therapeutically effective amount of the pharmaceutical composition of claim 1.

9. The method of claim 8 wherein said 5-HT_{1F}-specific agonist in said pharmaceutical composition is 2,4,6-trifluoro-N-[6-(1-methyl-piperidin-4-ylcarbonyl)-pyridin-2-yl]-benzamide.

10. The method of claim 9 wherein said NSAID in said pharmaceutical composition is present at 1-600 mg and is selected from the group consisting of: aspirin; ibuprofen; acetaminophen; naproxen; diclofenac; ketorolac; etodolac; flurbiprofen; oxaprozin; indomethacin; mefenamic acid; nabumetone; piroxicam; celecoxib; rofecoxib; valdecoxib; and lomoxicam.

11. A method of treating a patient for migraine, or headache pain, comprising:
a) administering to said patient an NSAID; and
b) administering to said patient a 5-HT_{1F}-specific agonist;
wherein said NSAID and said 5-HT_{1F}-specific agonist are administered in a co-timely manner and are administered in a therapeutically effective amount.

12. The method of claim 11, wherein said 5-HT_{1F}-specific agonist is administered within two hours of the NSAID.

13. The method of claim 12, wherein said NSAID has an onset of action of two hours or less.

14. The method of claim 11, wherein said NSAID and said 5-HT_{1F}-specific agonist are administered in an amount sufficient to provide for therapeutic synergy or migraine-related therapeutic synergy and wherein neither drug is administered in an amount sufficient to provide a therapeutic effect in the absence of the other.

15. The method of claim 11, wherein said NSAID and said 5-HT_{1F}-specific agonist are each administered at a dose of 1-600 mg.

16. The method of claim 15, wherein said NSAID and said 5-HT_{1F}-specific agonist are each administered at a dose of 10-400 mg.

17. The method of claim 15, wherein said 5-HT_{1F}-specific agonist is 2,4,6-trifluoro-N-[6-(1-methyl-piperidin-4-ylcarbonyl)-pyridin-2-yl]-benzamide.

18. The method of claim 17, wherein said NSAID is selected from the group consisting of: aspirin; ibuprofen; acetaminophen; naproxen; diclofenac; ketorolac; etodolac; flurbiprofen; oxaprozin; indomethacin; mefenamic acid; nabumetone; piroxicam; celecoxib; rofecoxib; valdecoxib; and lornoxicam.

19. The method of claim 15, wherein said NSAID is selected from the group consisting of: aspirin; ibuprofen; acetaminophen; naproxen; diclofenac; ketorolac; lornoxicam; and etodolac.

20. The method of claim 19, wherein said NSAID is administered at a dose of 5-600 mg.
